(19) United States
(12) Patent Application Publication
(10) Pub. No.: US 2023/0372233 A1
(43) Pub. Date: Nov. 23, 2023

(54) COMPOSITION AND METHODS FOR HAIR REGROWTH

(71) Applicant: Mirparm LLC, Woodland, CA (US)

(72) Inventor: Michael Mansur, Sacramento, CA (US)

(73) Assignee: Mirparm LLC, Woodland, CA (US)

(21) Appl. No.: 18/321,701

(22) Filed: May 22, 2023

Related U.S. Application Data
(60) Provisional application No. 63/344,996, filed on May 23, 2022.

(51) Int. Cl. A61K 8/9794; A61Q 1/00; A61K 8/9711; A61Q 17/00; A61K 8/19; A61K 8/9789; A61K 31/165; A61K 8/26; A61K 8/67; A61K 36/87; A61P 17/14; A61Q 5/00; A61Q 5/02; A61Q 19/10; A61K 33/06

(52) U.S. Cl. CPC A61K 8/9794; A61Q 1/00; A61K 8/9711; A61Q 17/005; A61K 8/19; A61K 8/9789; A61K 31/165; A61K 8/26; A61K 8/676; A61K 36/87; A61P 17/14; A61Q 5/002; A61Q 5/02; A61Q 19/10; A61K 33/06; A61K 2236/39

(57) ABSTRACT

Provided is a hair growth composition comprising capsaicin, a vegetable oil, activated charcoal, aluminum sulfate, saw palmetto, and kelp. A method of preparing the above hair growth composition is additionally provided. Use of the above hair growth composition for the manufacture of a medicament for the treatment of hair loss is also provided. A method of treating a subject suffering from hair loss is further provided.

### CROSS-REFERENCE TO RELATED APPLICATIONS

This application claims the benefit of U.S. Provisional Application No. 63/344,996, filed May 23, 2022, and is incorporated by reference herein in its entirety.

### BACKGROUND OF THE INVENTION

### (1) Field of the Invention

The present application generally relates to the field of hair loss and hair health. More specifically, the invention is directed to the restoration of hair growth and hair health with a topical hair growth composition treatment.

### (2) Description of the Related Art

The average human has about 100,000 hairs, each growing out from a follicle residing on the scalp. It has been long known that hair follows a specific growth cycle with three distinct and concurrent phases: (a) anagen, where 85%-90% of the hairs on one's head physically grow approximately 1 cm per month lasting from three to five years; (b) catagen, where the hair is cut off from its nourishing blood supply and pushed upward in about two weeks; and (c) telogen (also known as shedding phase), where 10%-15% of the hairs break free from the softened follicle root and fall off from one's scalp. A healthy human replenishes 50 to 100 hairs per day as part of this natural cycle.

Hair loss (alopecia or baldness) is a debilitating chronic condition, causing a great deal of psychological patient morbidity and decreased patient quality of life. Two potential causes for hair loss are disruption of normal hair growth cycle and damage to the hair follicle. Under such conditions, hair may begin to fall out more quickly than it is regenerated, leading to symptoms such as a receding hairline, hair falling out in patches, or overall hair thinning. Hair loss may be linked to a person's genetics, although many medical and behavioral conditions may interrupt the growth cycle and cause hair loss.

Common types of hair loss include androgenetic alopecia (AGA), alopecia areata, and telogen effluvium.

Androgenetic alopecia (AGA) is the most common type of hair loss, affecting more than 50 million men and 30 million women in the United States. There are two types of androgenetic alopecia: (a) male-pattern hair loss and (b) female-pattern hair loss. In male-pattern hair loss, the cause is a combination of genetics and male hormones. Hair loss can begin any time after puberty and progress over the course of years or decades. It starts above the temples and continues around the perimeter and the top of the head, often leaving a ring of hair along the bottom of the scalp. Many men with male pattern hair loss eventually become bald.

The cause of female pattern hair loss is not fully understood but strongly associated with genetics. Hair slowly thins all over the scalp, but the hairline usually doesn't recede. Many women experience this type of hair loss as a natural part of aging, although hair loss may begin any time after puberty. Female pattern hair loss can cause hair to thin dramatically, but only rarely does it lead to baldness. Around 40% of women will experience some type of hair loss before reaching years of age. Most women start to see hair loss in the form of a widening part, diffuse thinning all over or bald patches. Some women simply notice an increase in the amount of hair falling out. Androgenetic alopecia is a progressive hair loss over many years.

In addition to genetic predisposition to hair loss, other factors may also contribute to the hair loss. (i) Age, where aging can cause an increased number of hairs transitioning into the resting phase of hair growth, causing remaining hairs to become shorter and thinner over time, a phenomenon known as involutional alopecia. (ii) Changes in hormone levels, where childbirth and menopause are common causes of hair loss in women due to hormone imbalances surrounding these events. (iii) Harsh styling practices, where frequent use of heat styling tools such as blow dryers, curling irons and straighteners, can also damage hair and lead to hair loss. Even wearing tight styles like ponytails, braids or buns for extended periods of time can cause hair loss. This type of hair loss is known as traction alopecia. (iv) Chemical hair treatments, where the chemicals used in coloring, perms and relaxers can damage hair and cause it to break, leading to an overall appearance of thinning.

Alopecia areata is another type of hair loss. The cause of alopecia areata is autoimmune disease. Alopecia areata affects roughly 6.8 million people in the United States. In the majority of cases, hair falls out in small patches around the size of a quarter. However, alopecia areata rarely leads to a complete loss of hair on the scalp.

Telogen effluvium (a thinning of hair) is also a type of hair loss. The cause of telogen effluvium is typically a physically or psychologically stressful event, such as losing a lot of weight, surgery, anemia, illness, and pregnancy.

In addition to these common types of hair loss, hair loss may also be caused by certain medications including chemotherapy, HIV/AIDS, hypothyroidism, and malnutrition including iron deficiency, as well as fungal infection, lupus erythematosus, radiation therapy, and sarcoidosis.

Many types of hair loss treatments are currently available, including medication and surgery.

Two medications, minoxidil and finasteride, have been approved to treat hair loss. Minoxidil (Rogaine®) is an over-the-counter nonprescription medication and can help people regrow their hair or slow the rate of hair loss or both. Minoxidil is applied to the scalp skin once daily for women and twice daily for men to be most effective. Usually, it takes several months to tell whether the treatment can prevent further hair loss or start hair regrowth. The mechanism of action of minoxidil is not well understood. Scalp irritation is likely one of the common side effects reported.

Finasteride (Propecia) is another prescription drug, approved for men only. Taken as a pill daily, finasteride can slow down hair loss and promote new hair growth. It also takes several months to tell whether finasteride treatment can prevent further hair loss or start hair regrowth. Rare side effects of finasteride include diminished sex drive and sexual function and an increased risk of prostate cancer.

Hair loss can also be treated with surgery, including hair transplant surgery and laser therapy. Hair transplant surgery works by moving a patch of scalp with hair to a bald spot. More than one surgery may be needed if the patient would like to transplant a large area. Hair transplant is a highly invasive procedure, requiring a sedation medicine to ease discomfort. Additional risks include bleeding, bruising, swelling and infection.

Laser hair growth therapy is less invasive than transplant surgery. Laser therapy is FDA approved for medical treatment of the most common type of hair loss, AGA. This procedure irradiates photons in scalp tissues to invigorate circulation, where the photons are further absorbed by cells on the scalp to encourage hair growth.

In summary, hair loss is caused by a collective set of hair and scalp diseases, involving a range of genetic, endocrine, immune, and inflammatory processes, each of which calls for its own specific form of treatment. As a result, any treatment focusing on a single aspect or the most prominent pathogenesis is likely to fail. The complexity of the hair loss pathways requires a solution where multiple treatments act simultaneously on multiple aspects of the system, including the hair, the follicle, the scalp, and the underlying nourishing blood supply. There is thus a need to provide a non-invasive and cost-effective hair loss treatment having minimal side effects. The present invention enables the healing power of multiple natural ingredients and satisfies that need.

### BRIEF SUMMARY OF THE INVENTION

Provided is a hair growth composition for treating hair loss. The hair growth composition comprises capsaicin, a vegetable oil, activated charcoal, aluminum sulfate, saw palmetto, and kelp.

Additionally provided is a method of preparing the above hair growth composition. The method comprises extracting components from a first mixture of kelp, a vegetable oil, and an activated charcoal by heating the first mixture at a first temperature;
extracting components from a second mixture of a cayenne pepper, an aluminum sulfate, and a saw palmetto by heating the second mixture at a second temperature;
extracting components from a third mixture of a rose water and a lemon juice by heating the third mixture at a third temperature;
mixing all extracted components from the first mixture, the second mixture, and the third mixture to a combined extraction mixture, wherein the combined extraction mixture is stirred at a fourth temperature;
cooling down the combined extraction mixture at a fifth temperature; and
removing any solid portion in the cooled combined extraction mixture.

A method of treating hair loss is additionally provided. The method comprises applying the above hair growth composition to a skin area of a patient desiring hair growth on the skin area.

### DETAILED DESCRIPTION OF THE INVENTION

Solution: As used herein, the term “solution” refers to a composition of matter which is in a liquid state, free of solids and precipitations. The hair growth treatment composition is a solution.

Mixture: As used herein, the term “mixture” refers to a composition of matter which contains solids, liquids, gases, and/or precipitations. Prior to the filtration step, the hair growth preparation composition is a mixture.

Treatment area: As used herein, the term “treatment area” refers to an area to which the hair treatment solution is applied. Such an area typically includes an at least partially bald human head.

Negative side effects: As used herein, the term “negative side effects” refers to side effects which are harmful or undesirable to the subject of the treatment. Such side effects include, but are not limited to, a dry scalp, a burning scalp, or a rash.

Cosmetic: As used herein, the term “cosmetic” refers to a composition of matter which can provide improved qualities to skin. Such qualities include, for example, cleansing of the treatment area.

Formula, formulating and formulation: As used herein, the term “formula” refers to a list of ingredients in percentage by weight that have been standardized to consistently make a hair regrowth composition. As used herein, the term “formulating” and the term “formulation” refer to processing instructions of the list of ingredients in percentage by weight that have been standardized to consistently make a hair regrowth composition.

Pharmaceutically acceptable carrier: As used herein, the term “pharmaceutically acceptable carrier” refers to a diluent, adjuvant, excipient, or vehicle with which a compound is administered. Such carriers can be sterile liquids, such as water and oils, including those of petroleum, animal, vegetable, or synthetic origin, such as peanut oil, soybean oil, mineral oil, sesame oil and the like, polyethylene glycols, glycerine, propylene glycol, or other synthetic solvents. Water is a preferred carrier when a compound is administered intravenously. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. A compound, if desired, can also combine minor amount of wetting or emulsifying agents, or pH buffering agents such as acetates, citrates, or phosphates. Antibacterial agents such as a benzyl alcohol or methyl parabens; antioxidants such as ascorbic acid or sodium bisulfate; chelating agents such as ethylenediaminetetraacetic acid; and agents for the adjustment of tonicity such as sodium chloride or dextrose may also be a carrier. Methods for producing compounds in combination with carriers are known to those of skill in the art.

Therapeutically effective amount: As used herein, the term “therapeutically effective amount” refers to those amounts that, when administered to a particular subject in view of the nature and severity of that subject's disease or condition, will have a desired therapeutic effect, e.g. an amount that will cure, prevent, inhibit, or at least partially arrest or partially prevent a target disease or condition.

In some embodiments, a hair growth composition for treating hair loss is provided. The hair growth composition comprises capsaicin, a vegetable oil, activated charcoal, aluminum sulfate, saw palmetto, and kelp.

Capsaicin (8-methyl-N-vanillyl-6-nonenamide) is an anti-inflammatory compound to treat painful inflammatory conditions and to soothe any inflammatory issues associated with the scalp. Capsaicin triggers valloniid receptors in hair follicles and boosts hair growth. Capsaicin also stimulates hair growth by inhibiting substance P (a peptide). In a study, participants with alopecia showed hair growth in a 5-month period when they ingested capsaicin and isoflavone.

In various embodiments, capsaicin is provided in the fruits of plants in the genus Capsicum family, including cayenne peppers, jalapeno peppers, bell peppers, friggitello peppers, paprika peppers, serrano peppers, and other chili peppers.

In some embodiments, capsaicin is provided in cayenne peppers. In some of these embodiments, cayenne peppers are provided in the composition in the range of at least 1 to 30 parts of the cayenne pepper per 1000 parts of the formula.

In various embodiments, vitamins A, C, and E, and folate are also provided in the cayenne peppers.

Vegetable oil is rich in carbohydrates and essential nutrients that can repair damaged hair. Major components of vegetable oil include triglycerides, free fatty acids, tocopherols, phospholipids, squalene, linoleic acid, polyphenols, and phenolic alcohols. These ingredients work together to maintain scalp and hair health. Vegetable oil can also help extract hydrophobic compounds that would otherwise be discarded. Additionally, vegetable oil stabilizes the composition where many ingredients co-exist in the mixture, therefore ensuring the fillability of the preparation.

Any vegetable oil can be utilized in the composition. In various embodiments, the vegetable oil is palm oil, coconut oil, jojoba oil, avocado oil, almond oil, olive oil, castor oil, sunflower oil, grape seed oil, sesame oil or a combination thereof. In some embodiments, the vegetable oil is provided in the range of 20 to 100 parts per 1000 parts of the formula.

In specific embodiments, the vegetable oil is red palm oil, which promotes cell growth of the hair follicles, resulting in stronger hair. Red palm oil is a vitamin E rich antioxidant that maintains collagen. Red palm oil holds hair together and decreases hair loss and hair graying.

Red palm oil has a long history of being safely consumed and used for over 5,000 years. Red palm oil is taken and partially processed from the fruit of the oil palm tree, where the red color is due to the presence of residual carotenes like alpha-carotene, beta-carotene, and lycopene. Red palm oil contains 45% saturated fat with a high percentage of monounsaturated fat while coconut oil contains approximately 90% saturated fat. Monounsaturated fat, one of the healthy fats along with polyunsaturated fat, is a type of dietary fat. Monounsaturated fat can help lower LDL cholesterol (“bad cholesterol”) levels and develop and maintain cells.

Red palm oil holds numerous health benefits for our body and hair. Red palm oil is rich in vitamin E and that benefits the hair by promoting cell growth of the hair follicles, which results in stronger hair. Vitamin E also decreases graying of the hair and hair loss. The carotenes in red palm oil are also responsible for the growth and division of cells and will prevent the hair from becoming weak and brittle. Red palm oil maintains the collagen that holds hair together, and thus reduces hair loss by promoting the growth of stronger, thicker, healthier hair while simultaneously slowing the appearance of graying hair.

Research with human subjects showed a 38% increase in the number of scalp hairs after 8 months of palm fruit tocochromanol supplementation (146 mg/day). The number of hairs was increased in thinning areas of the scalp in both men and women suffering from hair loss. Both human clinical trials demonstrated a significant regrowth of hair on the scalp, without side effects.

Saturated fats and lipids are a perfect moisturizer for our skin and hair. Palm oil can be found in shampoos, conditioners, soap, lotions, and other beauty products because of its anti-aging properties and its ability to remove oil and dirt from skin and hair. Palm oil also contains a refatting agent that helps restore our hair and skin's natural oils that standard soaps and shampoos can remove. Palm oil also has antibacterial properties that will eliminate parasites, clean the skin and scalp, and create a healthier environment for hair growth.

Activated charcoal, also known as activated carbon, is an active powder that is a byproduct of carbon-containing materials. The activated charcoal is a fine, odorless, tasteless, non-toxic ingredient. It has pores, and it is negatively charged, which helps attract toxins. For that reason, activated charcoal is sometimes used in medical settings, including emergency poison treatment, for pumping the stomach after someone has ingested medications or poisons.

Activated charcoal has multiple benefits when used on the scalp and hair. Millions of pores in the scalp and hair can trap everything in close contact, from smoke and pollutants to chemicals from products or processing, bacteria, dirt, sludge, and toxins. The constant buildup of these harmful materials can clog hair follicles, and the clogged follicles often lead to inflammations and infections that are associated with hair loss. Activated charcoal draws out harmful materials from the scalp and the hair itself. Activated charcoal can absorb up to 1000 times its own weight in impurities, making it among the most effective cleaning agents you can use on your hair. Activated charcoal thus helps detoxify and clear scalp build-up.

Activated charcoal selectively lifts and eliminates certain materials from the scalp, root, and hair shaft, leaving the underlying structures intact. Materials that can be removed by activated charcoal include dirt, grime, toxins, pollutants, excess oil from product usage, excess sebum, dead or dry skin cells, sweat, and smelly impurities. Activated charcoal is antibacterial, fighting against microorganisms that cause scalp itchiness and irritations. Thus, activated charcoal cleans and restores a balanced and healthy environment around the scalp and hair.

Aluminum sulfate is a soluble salt with the formula Al₂(SO₄)₃ in water. Aluminum sulfate is a coagulating agent, promoting particle collision by neutralizing charge, and is used in the purification of drinking water and in wastewater treatment plants. Aluminum sulfate is acidic and the low pH helps smoothen and tighten the cuticle around the hair surface, resulting in shinier hair overall. Aluminum sulfate has antibacterial properties that can help scalp health and make hair healthier and stronger overall. Finally, aluminum sulfate is an astringent compound, which can stop small cuts from bleeding and further contribute to a balanced and healthy environment around the scalp and hair.

Saw palmetto is a small palm tree with large leaves and large deep red-black berries. Saw palmetto berries contain over 100 known compounds, including a variety of fatty acids and phytosterols. The active ingredients in saw palmetto are contained in the purified lipid soluble extract of the saw palmetto berry. Saw palmetto extract is a potent berry extract that targets the natural regulation of hormones that play a role in hair loss. Saw palmetto extract may block 5-alpha-reductase, an enzyme that converts testosterone to dihydrotestosterone (DHT). DHT is a molecule responsible for hair loss. In a 2012 study, 38% of patients treated with saw palmetto for two years saw improvements in their hair growth rate.

Saw palmetto extract may reduce inflammation of many scalp-related issues and hair growth problems, therefore improving the overall health of the scalp. A healthier scalp may lead to a healthier hair follicle, and then lead to stronger and thicker hair.

Kelp is rich in many of the key minerals and vitamins that our bodies require, including iodine, calcium, zinc, potassium, magnesium, iron, laminaria angustata, folate, natural fiber, alginate, vitamins A, B1, B2, C, D and E, and the essential amino acid 1-lysine.

Kelp boasts many significant benefits from anti-aging and hormonal balancing properties to protecting thyroid health. Some of the key nutrients in kelp are tied directly to hair health. Sea kelp, a specific type of kelp, is an extremely nutrient-dense plant having antioxidant benefits and help with hair loss. The iodine in sea kelp regulates the thyroid, and is good for dandruff and thinning hair. Several of the minerals in sea kelp help promote both a healthier scalp and stronger hair. Iron in sea kelp boosts energy levels and promotes the function of B vitamins that are vital for healthy skin, strong nails and hair growth. A study showed that seaweed eaters had a 95-100% hair regrowth rate while those that did not eat seaweed only had a regrowth rate of 25-30%. Sea kelp also helps the scalp and hair maintain proper hydration, therefore improving dry hair and giving shine to strands. Sea kelp has been added to shampoos, conditioner, and hair treatments for strengthening and nourishing hair and scalp.

In various embodiments, the composition further comprises rose water. Rose water is a long-lasting fragrant liquid that contains the essence of rose. Rose water is produced through steam distillation of fresh rose petals. It is colorless, aromatic and long-lasting.

Rose water contains anti-inflammatory and antioxidant materials. The anti-inflammatory materials soothe irritated skin while the antioxidant materials help to strengthen skin cells and regenerate skin tissue. Rose water also stimulates the circulation of tiny blood vessels underneath the skin. Furthermore, rose water assists in maintaining skin pH balance, and calms dermatitis and eczema. Rose water also hydrates and revitalizes the skin while healing scars, cuts, and wounds that may be caused by various natural factors such as accidents, as well as from chemical damage to the scalp. Additionally, rose water helps to hydrate and condition the hair shaft and helps make the hair shaft strong and flexible in order to help resist hair breakage.

Topically applying rose water on the scalp and hair can have many benefits. It is a mild scalp astringent that can prevent excess oil production on the scalp. Rose water is also a defense against dandruff, preventing dandruff formation on the scalp by reducing oil production and supplying a variety of vitamins. Rose water is also an anti-inflammatory agent that has natural antioxidants and anti-inflammatory properties that can soothe irritations on the scalp. Additionally, rose water is a frizz-fighting moisturizer, providing hydration to dry and curly hair. Further, rose water prevents hair loss by providing a healthy scalp environment.

In some embodiments, the composition further comprises a stabilizer, water, and a fruit or vegetable juice.

Juice is made from the extraction or pressing of the natural liquid contained in fruit and vegetables, including apple juice, beet juice, blueberry juice, cranberry juice, carrot juice, a citrus juice, grape juice, grapefruit juice, lemon juice, orange juice, pomegranate juice, tart cherry juice, tomato juice. Juice is rich in many nutrients as well as citric acid, calcium, magnesium, vitamin C, pectin, and flavonoids, each of which is beneficial for hair growth. Lemon juice contains nutrients that can strengthen the hair follicles and encourage new hair growth. Because of its naturally acidic property, lemon juice can deeply cleanse the scalp and hair follicles, restore healthy pH levels, and remove build-up of product, oils, and pollutants. Lemon juice also improves the strength of follicles and hair roots, naturally stimulating hair growth.

Vitamin C in lemon juice improves collagen production which in turn makes hair grow (see below). Citric acid in lemon juice prevents hair follicles from getting loose, thus reducing hair fall. Lemon juice also unclogs the pores around the follicles to reduce dandruff when used in the scalp. Additionally, lemon juice reduces the production of oil in the scalp, thus preventing an oily scalp and look. Also, lemon juice is anti-fungal, thus keeping the scalp infection free when used regularly.

In some embodiments, the composition of the present invention further comprises vitamin C and/or grape seed extract.

Vitamin C (also known as ascorbic acid and ascorbate with the molecular formula of C₆H₈O₆) is a water-soluble vitamin found in citrus and other fruits and vegetables. Vitamin C is a powerful antioxidant that helps protect against the oxidative stress caused by free radicals. Free radicals are naturally formed in energy production from glucose. Free radicals damage the hair by making it weak, brittle and thin, and can block hair growth and cause hair to age. To counter-react the damaging effect from free radicals in interrupting hair growth, vitamin C's antioxidant properties reduce the formation of free radicals and minimizes their damaging effect on the proteins in hair. The antioxidant vitamin C is also hygroscopic, attracting and binding water, locking moisture into the hair and preventing hair breakage and dulling.

Vitamin C also helps the body absorb iron, a mineral necessary for hair growth. Vitamin C is an essential nutrient for damaged hair and thus, treats and prevents a variety of hair disorders which can damage the hair follicles and affect the normal growth of hair. A diet containing significant levels of vitamin C can help combat alopecia and baldness in men. It also helps improve blood circulation and strengthens and repairs capillaries. This is particularly important for getting stronger, thicker hair. In addition, vitamin C is an intermediate compound for the body to synthesize collagen—an essential structural component of a strong and healthy hair.

Vitamin C also lowers the pH to create an acidic environment that helps fight the bacteria on the scalp. The acidic environment wards off dandruff and helps to get rid of the follicles' debris and encourages the growth of new hair. The acidic environment also helps with dry and itchy scalps because of its antiviral properties. Vitamin C also helps the body to clear follicles from clogging due to dandruff and dry, flaky skin.

Insufficient intake of vitamin C may be a root cause for a number of hair-related problems that affect hair growth. Vitamin C deficiency may result in dry hair and split ends. These conditions are unfavorable for the regular growth of hair.

Grape seed extract (GSE) is a valuable by-product from the grape juice and the grape vine industries. GSE has been recognized for its medical value and nutritional properties for more than 6,000 years. GSE is rich in antioxidant components flavonoids and stilbenes like resveratrol. GSE shows significant antibacterial activity against Gram-positive and Gram-negative pathogenic bacteria. GSE is able to inhibit DHT production, and thus prevent hair follicle loss. Because GSE is a potent ingredient that acts as an antiallergenic, antihistamine, anti-inflammatory, and antioxidant, GSE is able to maintain a healthy environment around the scalp and hair follicles.

In some of these embodiments, the composition contains at least 1 to 30 parts of cayenne pepper per 1000 parts of the formula, 20 to 100 parts of vegetable oil per 1000 parts of the formula, 1 to 10 parts of activated charcoal per 1000 parts of the formula, 0.5 to 3 parts of aluminum sulfate per 1000 parts of the formula, 0.1 to 15 part of saw palmetto per 1000 parts of the formula, and/or at least 10 to 90 parts of kelp per 1000 parts of a formula.

In additional embodiments, the composition contains at least 1 to 30 parts of cayenne pepper per 1000 parts of the formula, 20 to 100 parts of vegetable oil per 1000 parts of the formula, 1 to 10 parts of activated charcoal per 1000 parts of the formula, 0.5 to 3 parts of aluminum sulfate per 1000 parts of the formula, 0.1 to 15 part of saw palmetto per 1000 parts of the formula, at least to 90 parts of kelp per 1000 parts of a formula, and/or 600 to 950 parts of a water per 1,000 parts of the formula.

Extraction is a separation technique employed in many industries. The extraction process employs a wide variety of steps to separate and recover a part of ingredients from whole food or unprocessed raw materials by squeezing, mashing, crushing, or extracting with a solvent or water and then, heating to remove the residue. The purpose of the extraction process is to move or enrich or deplete one or more compounds of interest from the unprocessed raw materials.

In some embodiments, the composition comprises a first extract obtained by extraction from a first mixture of the kelp, the vegetable oil, and the activated charcoal; and a second extract obtained by extraction from a second mixture of the cayenne pepper, the aluminum sulfate, and the saw palmetto.

In some of these embodiments, the composition comprises a first extract obtained by extraction from a first mixture of at least 10 to 90 parts of kelp per 1000 parts of a formula, 20 to 100 parts of vegetable oil per 1000 parts of the formula, and 1 to 10 parts of activated charcoal per 1000 parts of the formula, and a second extract obtained by extraction from a second mixture of at least 1 to 30 parts of cayenne pepper per 1000 parts of the formula, 0.5 to 3 parts of aluminum sulfate per 1000 parts of the formula, and 0.1 to 15 part of saw palmetto per 1000 parts of the formula.

In further embodiments, the composition comprises a first extract obtained by extraction from a first mixture of at least 10 to 90 parts of kelp per 1000 parts of a formula, 20 to 100 parts of vegetable oil per 1000 parts of the formula, and 1 to 10 parts of activated charcoal per 1000 parts of the formula and a second extract obtained by extraction from a second mixture of at least 1 to 30 parts of cayenne pepper per 1000 parts of the formula, 0.5 to 3 parts of aluminum sulfate per 1000 parts of the formula, and 0.1 to 15 part of saw palmetto per 1000 parts of the formula.

In additional embodiments, the composition comprises a first extract obtained by extraction from a first mixture of at least 10 to 90 parts of kelp per 1000 parts of a formula, 20 to 100 parts of vegetable oil per 1000 parts of the formula, and 1 to 10 parts of activated charcoal per 1000 parts of the formula, a second extract obtained by extraction from a second mixture of at least 1 to parts of cayenne pepper per 1000 parts of the formula, 0.5 to 3 parts of aluminum sulfate per 1000 parts of the formula, and 0.1 to 15 part of saw palmetto per 1000 parts of the formula, and a third extract obtained by extraction from a third mixture of 600 to 950 parts of water per 1,000 parts of the formula, wherein the water is a purified water or a flower infused water (e.g., rose water).

Also provided is a method of preparing the above hair growth composition. The method comprises steps of extracting components from a first mixture of kelp, a vegetable oil, and an activated charcoal by heating the first mixture at a first temperature; extracting components from a second mixture of cayenne pepper, an aluminum sulfate, and saw palmetto by heating the second mixture at a second temperature; extracting components from a third mixture of a rose water, vitamin C and lemon juice by heating the third mixture at a third temperature; mixing all extracted components from the first mixture, the second mixture, and the third mixture to a combined extraction mixture, wherein the combined extraction mixture is stirred at a fourth temperature; cooling down the combined extraction mixture at a fifth temperature; and removing any solid portion in the cooled combined extraction mixture. The removal of the solid portion can be by any means known in the art, for example by centrifugation or by filtration, e.g., by gravity or by a Buchner funnel.

### Pharmaceutical Formulation and Route of Administration

General methods and compositions for dermal formulations are provided in Brown et al, 2019, and Walters, Kenneth A., 2002.

Drugs can be given to a person in a variety of formulations. Formulations is a mixture in a particular state that the drug is supplied to the patient. It can be either solid, semisolid or liquid formulations. The type of formulation given to a patient depends upon the type of patient and the condition of the patient (such as age, sex, and health condition). Also, the type of formulation is specific for particular routes of administration.

Dosage of the therapeutic agent(s) of the invention is dependent upon many factors including, but not limited to, the severity of the hair loss, the subject's age, general health and individual response to the compositions of the invention. Accordingly, dosages of the compositions can vary and be readily adjusted, depending on each subject's response.

In some embodiments of formulating the above composition, the composition comprises a pharmaceutically acceptable stabilizing agent, an anti-oxidant, a preservative, a buffering agent, a chelating agent, a tonicity agent, or any combination thereof.

The above-described composition can be formulated into any form. Nonlimiting examples include a solid formulation, a semi solid formulation, a liquid formulation, a foam formulation, a powder, an ointment, a foam, a lotion, a cream, an emulsion, a paste, a gel, an aqueous solution, a spray, a patch, a serum, a soaked gauze, a dressing, or any combination thereof.

In some embodiments, the hair growth composition is applied to a skin area of a patient desiring hair growth on the skin area, for example the scalp of the patient. In some of these embodiments, the patient has androgenic alopecia, male pattern hair loss, female pattern hair loss, or alopecia areata.

Preferred embodiments are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### Example 1. Preparation of Hair Growth Formula

This hair growth formula works especially well on women, while working on men as well. Within two to three weeks, or less, it becomes obvious that individuals who have tried it are growing hair on their scalp, with thicker, stronger, faster growing hair, often also described as shinier. This can include hair lines moving to that of a persons' earlier and fuller hair. There is also hair growth where hair was not growing previously. The overall quality of their hair growth is also vastly improved. It is noted that we are not finding hair growth in unwanted places, such as arms or hands.

Containers for mixing, manufacturing, and packaging of the formula may be selected from inert materials made of glass, metal, or silicone. Plastic containers may be used in some embodiments, however the effectiveness of the formula may be reduced as a result of the poor quality of some plastic containers.

The “Part” in the unit is defined by weight. For example, one part material X per 1,000 parts of the formula is equivalent to 1 g material X per 1000 g of the formula.

Any additions to the formula made after filtration are counted as a percent of the resulting hair growth solution.

One process for preparing the formula is separated into Phase A, Phase B, and Phase C.
Phase A: At least 10 to 90 parts kelp per 1,000 parts of the formula, preferably flocculent; to 100 parts of vegetable oil per 1,000 parts of the formula, preferably palm oil and most preferably red palm oil; and 1 to 10 parts of activated charcoal per 1,000 parts of the formula, preferably flocculent are mixed in a container and are heated to 45° C.-85° C. for ½ to 7 hours.
Phase B: 1 to 30 parts of cayenne pepper per 1,000 parts of the formula; 0.5 to 3 parts of aluminum sulfate per 1,000 parts of the formula; and 0.1 to 15 parts of saw palmetto per 1,000 parts of the formula, mixed and heated to 50° C.-95° C. for ½ to 5 hours.
Phase C: 600 to 950 parts of water per 1,000 parts of the formula, preferably flower infused water, most preferably rose water; and up to 200 parts of juice per 1,000 parts of the formula, preferably lemon juice, mixed in a container and are heated to 25° C.-75° C. for at least 1 hour.

Finished ingredients in Phase B is then added to finished ingredients in Phase A and mixed for at least 3 minutes. The resulting finished ingredients mixture in Phase A and Phase B are then added to finished ingredients in Phase C and mixed for at least 3 minutes. This mixture is then heated to 25° C.-75° C. for ½ to 6 hours, followed by a refrigeration step at 2° C.-25° C. for at least 4 hours, where solids can be observed.

A filter paper, e.g., Whatman number 1 filter paper, is then used to filter out the resulting solids from the liquid portion of the mixture by simple gravity or under vacuum (e.g., through a Buchner funnel), while keeping the filtration assembly refrigerated at 2° C.-25° C.

Collect the “watery” liquid solution from the filtration step and add an effective amount of potassium sorbate as a preservative for long term storage.

### Example 2. Effectiveness of the Hair Growth Formula

A woman began using the formulation described in Example 1 in her mid-forties and used the invention over a period of more than 5 years. At the outset one could easily see her scalp skin through her hair. Throughout her use of the product, she continuously had new growth and thickness in her hair. The hair appeared to fill in more and more. She got comments from people that had known her for a long time about how good it looked. Many pictures she took over the hair growth formula treatment showed a big difference from a year ago. She saw new growth all the time and the health of her hair appeared to be vibrant. The formula was easy to use and left her hair soft and shiny and feeling good. She wanted to say that she really appreciated the opportunity to be a part of this hair growth formula treatment and looked forward to continuing to use it.

### Example 3. Effectiveness of the Hair Growth Formula

A female subject was given the hair growth formula described in Example 1. The subject's hair stylist had used the hair growth formula described in Example 1 earlier and was successful. The subject's hair stylist then suggested that the female subject try the hair growth formula as the female subject had always had fine thin hair. After the female subject used the hair growth formula over a period of time, this hair growth formula really worked and the female subject called the hair growth formula her “magic hair tonic”. The female subject had tried every thinning hair regrowth and thickening product and extension system out there prior to using this hair growth formula, but nothing else worked like this hair growth formula. After the female subject started using the hair growth formula, the female subject saw results almost immediately. Within three months the female subject had baby hair growing and the female subject's hair became fuller in places that had not seen growth in years. With this extra growth, it gave the female subject added strength to grow hair out longer than it had ever been. The female subject stated that the hair growth formula was AMAZING!

### Example 4. Effectiveness of the Hair Growth Formula

A 21-year-old female hairdresser was given the hair growth formula. After she used a single six-week supply, she then reported the invention was the only hair growth application that ever worked like the way someone promised it would. During the course of the single six-week supply, she saw her hair grow by an inch and a half, as opposed to its usual growth over that period of a half inch. She noticed the difference in a week from the beginning of the application of the invention and she also noted that her hair was stronger and shinier than before her use of the invention. She also reported seeing “new root hair” during the course of her applying the invention.

### Example 5. Effectiveness of the Hair Growth Formula

This example documents the inventor's personal experience with the hair growth formula. After the inventor created the formula in the laboratory, he first used it in the latter part of August 2006. Within ten days of first use of the hair growth formula, an inventor's friend commented on seeing more hair on the inventor's head. Though the inventor was never completely bald, the hair had become sparse and thin particularly on top of the head. It was easy to see through to the scalp before the inventor used the hair growth formula. Now after using the hair growth formula for more than 15 years, there was more hair on his head than there was then, and the individual hairs were thicker.

### REFERENCES

Brown, Marc B., and Adrian C. Williams. The Art and Science of Dermal Formulation Development 2019 (Drugs and the Pharmaceutical Sciences). CRC Press, 2019
Walters, Kenneth A. Dermatological and Transdermal Formulations 2002 (Drugs and the Pharmaceutical Sciences). CRC Press, 2002

In view of the above, it will be seen that several objectives of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification, and references cited therein, are hereby incorporated by reference. The discussion of the references herein is intended merely to summarize the assertions made by the authors and no admission is made that any reference constitutes prior art. Applicants reserve the right to challenge the accuracy and pertinence of the cited references.

As used herein, in particular embodiments, the terms “about” or “approximately” when preceding a numerical value indicates the value plus or minus a range of 10%. Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure.

The indefinite articles “a” and “an,” as used herein in the specification and in the embodiments, unless clearly indicated to the contrary, should be understood to mean “at least one.”

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements can optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements can optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

What is claimed is:

1. A hair growth composition for treating hair loss or improving hair health, the hair growth composition comprising capsaicin, a vegetable oil, activated charcoal, aluminum sulfate, saw palmetto, and kelp.

2. The hair growth composition of claim 1, further comprising a stabilizer, water, lemon juice, vitamin C, grape seed extract, or any combination thereof.

3. The hair growth composition of claim 1, wherein the vegetable oil is red palm oil.

4. The hair growth composition of claim 1, wherein the activated charcoal is flocculation optimized.

5. The hair growth composition of claim 1, wherein the capsaicin is in cayenne pepper.

6. The hair growth composition of claim 1, comprising
a first extract obtained by extraction from a first mixture of the kelp, the vegetable oil, and the activated charcoal; and
a second extract obtained by extraction from a second mixture of the cayenne pepper, the aluminum sulfate, and the saw palmetto.

7. The hair growth composition of claim 6, wherein:
the first mixture contains at least 10 to 90 parts of the kelp per 1000 parts of a formula, 20 to 100 parts of the vegetable oil per 1000 parts of the formula, and 1 to 10 parts of the activated charcoal per 1000 parts of the formula; and
the second mixture contains at least 1 to 30 parts of the cayenne pepper per 1000 parts of the formula, 0.5 to 3 parts of the aluminum sulfate per 1000 parts of the formula, and 0.1 to 15 parts of the saw palmetto per 1000 parts of the formula.

8. The hair growth composition of claim 1, further comprising a mixture of 600 to 950 parts of a water per 1,000 parts of the formula, wherein the water is a purified water or rose water.

9. The hair growth composition of claim 1, further comprising:
a third extract obtained by extraction from a third mixture of up to 200 parts of lemon juice per 1,000 parts of the formula.

10. The hair growth composition of claim 1, further comprising a pharmaceutically acceptable stabilizing agent, an anti-oxidant, a preservative, a buffering agent, a chelating agent, a tonicity agent, or any combination thereof.

11. The hair growth composition of claim 1, wherein the hair loss is androgenic alopecia, male pattern hair loss, female pattern hair loss, or alopecia areata.

12. The hair growth composition of claim 1, formulated as a powder, an ointment, a lotion, a cream, an emulsion, a paste, a gel, an aqueous solution, a spray, a patch, a serum, a soaked gauze, a dressing, or any combination thereof.

13. A method of preparing the composition claim 1, the method comprising
extracting components from a first mixture of kelp, a vegetable oil, and an activated charcoal by heating the first mixture at a first temperature;
extracting components from a second mixture of a cayenne pepper, an aluminum sulfate, and a saw palmetto by heating the second mixture at a second temperature;
extracting components from a third mixture of a rose water and a lemon juice by heating the third mixture at a third temperature;
mixing all extracted components from the first mixture, the second mixture, and the third mixture to a combined extraction mixture, wherein the combined extraction mixture is stirred at a fourth temperature;
cooling down the combined extraction mixture at a fifth temperature; and
removing any solid portion in the cooled combined extraction mixture.

14. The method according to claim 13, wherein the solid portion in the cooled combined extraction mixture is coalesced and the solid portion is removed by membrane filtration or centrifugation.

15. The method of claim 13, wherein:
the first temperature is between 45° C. and 85° C.;
the second temperature is between 50° C. and 95° C.;
the third temperature is between 25° C. and 75° C.;
the fourth temperature is between 25° C. and 75° C.; and
the fifth temperature is between 2° C. and 25° C.

16. The method of claim 13, wherein:
the first mixture is heated for between 0.5 hours and 7 hours;
the second mixture is heated for between 0.5 hours and 5 hours;
the third mixture is heated for at least 1 hour;
the combined extraction mixture is stirred; and
the combined extraction mixture is allowed to cool at least 4 hours.

17. The method of claim 13, wherein the combined extraction mixture is achieved in sequential order, wherein:
the second mixture is first added to the first mixture; and
the third mixture is added to the combined first mixture and second mixture.

18. The method of claim 13, further comprising adding a pharmaceutically acceptable stabilizing agent, anti-oxidant, preservative, buffering agent, chelating agent, tonicity agent, or any combination thereof.

19. A method of treating hair loss, the method comprising applying the composition of claim 1 to a skin area of a patient desiring hair growth on the skin area.

20. The method according to claim 19, wherein the patient has androgenic alopecia, male pattern hair loss, female pattern hair loss, or alopecia areata.
